(19) United States
(12) Patent Application Publication
(10) Pub. No.: US 2007/0059782 A1
(43) Pub. Date: Mar. 15, 2007

(54) Magnetic particle tagged blood bank reagents and techniques

(76) Inventors: Henry A. Graham, Solana Beach, CA (US); John G. Gorman, Del Mar, CA (US); James P. Rowell, Stockton, NJ (US)

Correspondence Address: RALPH T. LILORE;ATTORNEY AT LAW, 371 FRANKLIN AVENUE, NUTLEY, NJ, 07110, US

(21) Appl. No.: 11/518,189

(22) Filed: Sep. 11, 2006

Related U.S. Application Data
(60) Provisional application No. 60/716,591, filed on Sep. 13, 2005.

(51) Int. Cl. G01N 33/567; G01N 33/551

(52) U.S. Cl. 435/7.21; 436/524

(57) ABSTRACT

Magnets and magnetic particle-labeled reagents are used to capture and/or release magnetic particle-tagged entities for immunohematology diagnostic testing purposes, especially tests performed in blood banking. The magnetic tagged entities may be tagged antibodies, tagged blood cells, tagged universal binding partners, especially tagged lectins and tagged Coombs reagent, and other binding agents such as biotin-avidin, Protein A or G, ligands and their receptors and the like. Separation of unbound material from bound material is effected through the use of one or both the magnetic field effect on the magnetic labeled reactants and the density gradients of layers of an assay construct. Constructs such as chromatographic strip lateral flow format, and liquid phase reactions in suitable vessels with end point determinations that do not require centrifugation to detect reacted entities. Readable labels such as enzymes, fluorophors, chemiluminescent materials, radioactive isotopes, and other labels may be attached to Coombs reagent to provide a readable product of the Coombs reagent with any antibody participating in the assay.

### CROSS REFERENCE TO RELATED APPLICATIONS

This application claims the benefit of U.S. Provisional Application 60/716,591, filed Sep. 13, 2005.

### STATEMENT REGARDING FEDERALLY SPONSORED RESEARCH OR DEVELOPMENT

(Not Applicable)

### REFERENCE TO A SEQUENCE LISTING, A TABLE, OR A COMPUTER PROGRAM LISTING APPENDIX SUBMITTED ON COMPACT DISC (SEE 37 CFR 1.52(e)(5))

(Not Applicable)

### FIELD OF THE INVENTION

This invention relates to blood banking immunological diagnostic testing and immunohematology and more particularly to blood cell serological testing using magnetic particles and magnets to separate bound entities to be measured from unbound entities. It also relates to the use of chromatographic media and liquid phase media in such measurements. It further relates to determination of antigens, antibodies and other proteins on blood cells, in serum and other bodily samples and the use of buffers and other fluids in the determination process.

### BACKGROUND OF THE INVENTION

### Blood Bank Reagents and Techniques

THE DIRECT COOMBS (Antiglobulin) TEST: The direct Coombs (antiglobulin) test, which is used in the investigation of anemias, will demonstrate whether red blood cells are coated with incomplete antibody, especially that of babies born to Rh-negative mothers. It will reveal whether antibodies have been adsorbed on the surface of the red cells while the baby was in the uterus and is important in diagnosing Rh hemolytic disease of the newborn. The direct Coombs (antiglobulin) test is performed by washing the red blood cells to be tested and attempting to agglutinate them with Coombs (antiglobulin) reagent. The Coombs reagent is widely available. This test, as well as the indirect test described below, are variously referred to herein as Coombs test, anti-globulin test, AHG test or variations thereof. The serum is variously referred to as Coombs serum, anti-human globulin serum, AHG serum or the like.

THE INDIRECT COOMBS (Antiglobulin) TEST: The indirect Coombs (antiglobulin) test is used to screen the patient's serum for atypical antibodies such as Rho (D), Kell (K), Duffy (Fya), and hr′ (c). The presence of any of these atypical antibodies can cause hemolytic disease of the newborn or transfusion reactions.

In the indirect test, an unknown serum is tested with human group O reagent red blood cells. Group O reagent antibody screening cells are available commercially. They are a group of two or three O Rh positive and Rh negative donor red blood cells selected so as to be positive on at least 50% of the cells for each of the common clinically important red blood cell antigens. If a serum gives a positive reaction with such screening cells, tested separately or as a mixture, it must contain an atypical antibody of unknown identity. The techniques involved in performing the direct and indirect antiglobulin and the reasons therefore, are well-known in the art.

ABO GROUPING: Red cell (forward) typing with anti-A or anti-B reagents will demonstrate the presence or absence of A and B antigens on the red cell. Serum (reverse) typing with reagent A and B red cells will demonstrate the presence of anti-A and anti-B in the serum.

OTHER REAGENTS USEFUL IN ABO GROUPING: Other reagents may be used routinely in ABO grouping. They are often essential for resolving discrepancies between forward and reverse typing. Blood is not usually released from the blood bank for transfusion until any such discrepancies have been resolved. Anti-A, B (Group O serum) can detect weak A variants that may be missed by regular anti-A reagent. Other reagents: Anti-A, B reagent (Group O serum), Anti-A₁ reagent (absorbed B serum or Dolichos lectin), Anti-H lectin (Ulex), Reagent O Rh-positive screening cells, Reagent A₂ cells

COMPATIBILITY TESTING: Crossmatch (compatibility) tests are performed to determine the suitability of the donor's blood for the particular recipient. Blood transfusions are not given before performing a major crossmatch to test the donor's red cells against the serum of the recipient. If both donor and recipient are of the same blood group, a minor cross-match may be done to test the recipient's red cells against the donor's serum. The minor crossmatch is of no value when donor and recipient belong to different blood groups because agglutination will occur. Major Crossmatch involves mixing donor's red cells with recipient's serum, centrifuging at 37° C. and adding antiglobulin reagent. Minor Crossmatch involves mixing donor's serum with recipient's red cells, centrifuging at 37° C. and adding antiglobulin reagent.

RH TYPING: The crossmatch makes it possible to avoid hemolytic transfusion reactions following a particular transfusion. Blood banks are also concerned about isosensitization. If, for example, a blood bank selects Rho (D)-positive blood for an Rho (D)-negative woman, she will not have an incompatible crossmatch or a transfusion reaction if she has no anti-Rho (D) antibodies in her blood, but she may become sensitized to the Rho (D) antigen. Initiation of the immune response presents problems for subsequent transfusions and for subsequent pregnancies if she has an Rho (D)-positive mate. Rho (D) negative donors, Rho (D)-negative women and their Rho (D)-negative mates, and Rho (D)-negative cord bloods are tested for the presence of Rho\variant (DU) antigen that may not always be detected by the anti-Rho (D) slide test. Various Rh typing methods and the appropriate controls are well known to the art.

ANTIBODY TESTS: Screening for antibodies is especially important for patients receiving blood and the obstetrical patient. In obstetrical patients, early detection allows time to prepare for possible intrauterine or exchange transfusion in cases of Rh hemolytic disease of the newborn. Once the presence of an antibody has been detected, the problem of its identification remains, but this has been simplified by the development of antibody identification panels of group O reagent red cells. These screening and identification methods are well known to those skilled in the art.

### SUMMARY OF THE INVENTION

The foregoing tests and other similar tests are traditional and conventional and performed routinely in the blood banking field. The present invention is useful in performing virtually all of such tests that are performed in the blood bank involving reactions between binding partners such as immunological binding partners or universal binding partners such as lectins, biotin-avidin, Protein A or G, ligands and their receptors and the like.

In the present invention, magnets and magnetic particle-labeled reagents are used to capture and/or release magnetic particle-tagged entities for immunohematology diagnostic testing purposes. The magnetic tagged entities may be, depending on the particular assay, any of tagged antibodies, tagged blood cells, tagged universal binding partners, especially red blood cells, binding agents such as lectins, biotin-avidin, Protein A or G, ligands and their receptors and the like.

More particularly, the invention utilizes magnetic particles directly labeled with antibody (such as anti-A, anti-B, anti-D or anti-human serum). With such reagents used in the assays of the invention, the red cells will only react with magnets if the red cells have the reactive antigen corresponding to the specific antibody on them (and in the case of anti-human serum have been washed clean of serum). In these assays the presence of an RBC on a magnet, is a positive event for the presence of the antigen sought and can be seen because of the hemoglobin in the cells.

Another reagent used in the invention are magnetic particles labeled with a red cell binding partner, i.e., a lectin or other universal red blood cell binding material (in effect an anti-RBC). The lectin or other binding molecule should be able to bind magnetic particles to all human red blood cells regardless of blood group, and must not react with Coombs serum or other human antibodies. The magnetic particles are used to move the RBCs through zones or are positioned at a location on a chromatographic strip so that fluids can move by the cells (i.e., the fluids move over the comparatively stationary cells). In this more universal format a labeled AHG reagent, not bound to a magnet, but labeled with a detectable indicator such as an enzyme, fluorophor, and the like, described in more detail below, is used to react with the magnet bound red cell complex and any bound serum antibody.

An essential part of the invention in one of its aspects, is to provide for separation of unbound material from bound material, wherein the separation is effected through the combined use of the magnetic field effect on the magnetic labeled reactant and the density gradients of layers in the path of flow of reagents. This is especially true of any test that utilizes the Coombs (anti-globulin reagent). The invention in another of its aspects provides a chromatographic lateral flow format with an end point determination that does not require centrifugation to detect reacted entities. Virtually all current blood bank diagnostic tests require centrifugation at some point. In another aspect, a liquid format is employed wherein the bound magnetic reagents are pulled through liquid layers of varying density gradient layers via magnetic field effect while the unlabeled material remains in a liquid phase of lower density.

The invention also may employ software to sense the progress of process to provide feedback to timing of incubation, reagent dispensing, order, amount of reagent dispensing, application or removal of magnetic field and the like.

### DETAILED DESCRIPTION OF THE INVENTION

The present invention provides novel reagents tagged with magnetic particles, first among which is anti-human globulin (AHG) (Coombs) reagent, tagged with magnetic particles. Red cells that react with this reagent by forming a complex with the AHG will be captured by the magnetic field of the invention. This “capture” will be an indication of the presence of antibodies bound to antigens on the surface of blood cells, such as red blood cells, white blood cells, platelets, and the like.

The invention also provides for novel magnetic particle tagged blood cell antibody reagents such as anti-A, anti-B, anti-D, anti-K etc and novel magnetic particle tagged blood cells, reagents such as the A and B antigens and red blood cells. These reagents comprise the usual blood typing reagents of various specificities as used in blood bank laboratories but tagged with magnetic particles or beads, so that red cells (or any other blood cells) coated with the antibody reagents and sample antibody coated onto tagged reagent red cells will be captured by the magnetic field of the invention and thus indicate the presence of the specific antigens on the blood cell surface corresponding to the specific antibody of the tagged particle complex. In addition, novel magnetic tagged reagents comprising entities such as lectins, non-immunological binding pairs and universal binding agents which will bind to all blood cells, regardless of blood type, may also be produced. Such blood cells, preferably, red blood cells, coated with these reagents by reaction therewith will be captured by the magnetic field of the invention and may be held stationary for reading or for further processing in the test such as washing, concentration, prolonging and enhancing antibody incubation etc. If the magnetic field is removed, the cells will continue to flow according to the individual test protocol.

Various indicator labels such as enzymes and their substrates, fluorophors, chemiluminescent materials, radioactive isotopes, and other labels may be attached to the appropriate reagents, especially to non-magnetic tagged anti-human globulin, and analyte proteins in the manner well-known in the immunoassay art and thus serve to act as the element by which the progress and results of the test may be observed and measured during the test run. The magnetic reagents can be prepared by methods well-known in the art. Magnetic particles attached to proteins have been made in the art and are well-known. Their process of preparation is well-known. When such labels are used in the method of the invention as a means to visualize the end point, suitable substrates or exciting media should be supplied at appropriate places in the assay construct.

Where desired, the magnets can be configured in various patterns to re-footprint flowing antibodies, red cells and reagents. The patterns could be alphabetic letters or words, circles, straight lines or artistic patterns of magnetic dots or other patterns such as indentations in the magnet surface to create a very recognizable picture of blood cell capture like a lithograph. Detection of the captured magnetic entity can be aided by computer algorithms for pattern recognition, if desired.

Magnetic strengths across the magnetic footprint can be varied allowing measurement of the strength of reaction and quantitation of antibodies by measuring how far migration takes place in an increasing magnetic field. This enables further fine separation of reactants. The use of magnets with gaps in the magnetic field so that some of the magnetic decorated blood cells can flow past through the gaps to be captured further onto the magnet to make a recognizable footprint pattern or for supplementary processing further along the chromatographic medium is also an aspect of the invention.

Software can be developed to process the video or still image of multiple test runs, to segregate tests being run in parallel or otherwise closely juxtaposed areas of chromatographic media. This technology is art-known for pattern recognition for monitoring flow, archiving test run images and interpretive data, processing and presenting clinical and workflow data generated in test runs.

In some tests, where it may not be practical to magnetically pre-tag patient's or donor's sample, it is useful to use an “indirect” test. In this test, we allow the analyte antibodies to react with the blood cells and then detect whether they have done so with AHG reagent tagged with magnetic particles as described above.

The following is illustrative of a blood bank test either on a lateral flow chromatographic strip or in a liquid phase system, employing a magnetic separation. For ease of reference, the test is described in a liquid system, but is equally applicable to lateral flow chromatography strips:

A mixture of reagent red blood cells, patient anti-serum and magnetic particle tagged lectin is prepared and allowed to incubate in a suitable assay construct. During the incubation, any antibody reactable with reagent red blood cells will react to result in a RBC coated with antibody. The magnetic lectin will react with the red blood cell since it is a universal RBC reactant. A magnetic field is applied to the reacted mixture (the complex) which is pulled by the magnetic field through a liquid zone in the strip and washed in that zone. It is then drawn by the magnet through a zone containing a layer of antihuman globulin (AHG) labeled with a detection label, such as an enzyme, fluorophor, chemiluminescent material, radioactive isotope or the like. As the magnetic tagged complex is drawn through the labeled AHG via the force of the magnet, the AHG combines with the antibody of the complex thus attaching a readable label to the magnetic complex. Separation of unreacted AHG from the magnetic complex is achieved by drawing the complex through liquid zones of increasing density, so that lighter unreacted labeled AHG will remain in the lower density regions and will not take part in the reading determination. The reading of the labeled AHG is facilitated by providing a suitable substrate for the label at the reading zone of the assay construct.

The effect of the magnetic pull-through is enhanced by providing layers of increasing density gradient as the flow goes through the various zones. By this means, debris and unreacted material will remain in areas appropriate to their densities whereas the heavier complexed materials will be drawn through to the layers of higher density.

In these tests, it is helpful to have a movable magnet to facilitate the movement of the reacted complex. It should be noted that the foregoing reactions apply to the determination of any blood constituent by the appropriate selection of reagents and magnetic and tagged particles. It should be apparent that the foregoing reaction scheme does not depend upon separation of bound magnetic particles from unbound magnetic particles, but rather on the separation of the bound labeled AHG from unbound labeled AHG.

In the chromatographic strip format, magnetized coated, reacted RBCs can be stopped on the magnet and immobilized as described above. Driving fluids or buffer solutions can be introduced onto the strip and allowed to flow by capillary action across the immobilized magnetic complex. This will separate the unbound labeled AHG from the bound labeled AHG and thus eliminate the possibility of unreacted AHG entering into the reading step.

The foregoing enables a single Mag reagent for red cell phenotyping including forward ABO typing. The magnetic particle lectin anti-RBC or magnetic particle RBC binding partner reagent described above enables a method for performing all blood serology tests with the single magnetic labeled antiglobulin reagent, and using only regular non-Mag reagents in addition. This eliminates the need to develop a whole series of magnetic reagents of different specificities in the preferred embodiment. This method allows all blood bank red cell test to be done with a single magnetic particle reagent and a labeled anti-globulin reagent.

Signal acquisition may be achieved by methods normally used in the art in addition to visual observation of accumulated red blood cells or scanning the test construct or viewing the construct with a CCD video camera with appropriate pixel processing software. Interpretation of streaming color patterns could also provide an analyte measure.

Separation of proteins from RBC by sedimentation in chromatographic strip media may be based upon differential specific gravity of RBC and proteins and selection of differing specific gravities or densities of the medium as will be described below. In strip chromatography, different gradients may be supplied by varying the density of layers of the chromatographic strip. As chromatographic strip membranes, any membranes used in the art of immunoassay may be employed as long as it does not non-specifically bind to the red cells or the magnetic particles or can be treated to remove non-specific binding characteristics. For example, typically used in the field are nitrocellulose, filter paper, fiberglass and the like. For most preferred results, porosities of greater than about 20 microns are preferred to allow the antibody complexed tagged RBC to settle (or be forced by the magnetic field) into the lowermost layer of the strip, as well be seen below.

As a separate method for direct typing, in the chromatographic strip method, MAG tagged reagent antibodies could be introduced with the red cells over the magnet and capture red cells coated with cognate antigen. In another separate method for anti-globulin testing, if antibody coated red cells are separated from serum proteins, for example by density, MAG-Coombs serum lying on the magnet will capture coated red cells as they flow past. Non-coated will not be captured.

When the magnetic particles are coated with typing antibodies or antiglobulin reagent, the simplest and easiest way to measure the presence and quantity of red cells bound to the magnetic reagents either in flow or stopped on a magnet is a densitometer scan reading through hemoglobin wavelength filter. An alternative is a CCD color camera with pixel assessing software. In certain situations, chemical amplification of the hemoglobin signal using a chromagen. e.g. tetramethyl benzidine (TMB) chromagen/substrate solution would be worthwhile. Enzyme amplification is another method of use. Often, visual observation is suitable for qualitative determination

In another embodiment, MAG tagged reagent red cells could be introduced just before or over the magnet and held waiting to capture cognate antibody(s). The MAG tagged reagent red cells are introduced in front of the flowing antibody because they flow more slowly when they are retarded by the magnetic field and will bind faster flowing cognate antibody(s) coming past them.

Because the flow or movement of red cells tagged with magnetic particles and serum antibodies analytes of a specimen flow at very different rates in a chromatographic medium in the presence of a magnetic field, it becomes the ideal way to separate them for testing them individually. No preparative centrifugation is necessary, the red blood cells being held in place as materials are removed or added by capillary flow.

Because of the great sensitivity of the magnetic method, red cells from multiple donors may be pooled and tested at the same time in the same test run. Blood cells from incompatible donors would be captured on the magnet anti-human globulin particles. An estimate of the number of incompatible donors would be made by quantitating the capture signal relative to the whole signal. Further work would have to be done to determine which donors were incompatible. This would enable a blood bank, instead of cross-matching blood units one by one for a patient, to crossmatch for each patient, in a same test run, a pool of donor units made from the entire stock, for example, of O positive units, in the blood bank refrigerator.

METHODS FOR QUANTIFYING A POSITIVE CROSSMATCH RESULT: A measurement of all cells present in the antiglobulin test phase compared to the number of positive cells collected on the magnet will allow the calculation of the number of units that are reactive with a patient's serum during a pooled crossmatch. This information will provide the technologist with critical information relative to the choice of reflex testing to efficiently find compatible blood for the patient. Using algorithms similar to those used when screening pools of up to 50 plasma donors for infectious disease markers, donor blood can be tested in smaller pools to determine which units are actually incompatible with the donor without cross-matching all of them, saving time, labor and materials.

The overall concept of the invention is to provide a device having at least two layers in the form of a layered construct such as a chromatographic strip or layers in a liquid system wherein said layers are of different densities. This establishes a density gradient across the construct in which the RBCs mix with antibodies as they settle, and are then separated from serum IgG by allowing them to continue into a stable zone of higher specific gravity. The strip or liquid system comprises magnetic particle-tagged anti-globulin (AHG) reagent, if needed. The following illustrates a test device for performing a number of blood bank tests using the differential density gradient concept of the invention. It may be desirable to add a thin layer of an intermediate specific gravity material before adding the reagents to Zone 1.

Presented below is a generic schematic of a device useful in the invention. It may be a chromatographic strip or may represent layers of liquids of different densities, in for example, a microtitre plate well. Because of the large number and great variety of combinations and permutations of reactants and reaction schemes, the device is presented in connection with particular reference to the procedural aspects of the device. Those skilled in the art will easily be able to adapt the construct to whatever assay they desire to perform.

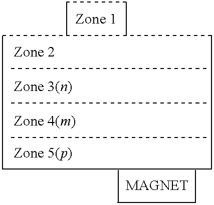

In the foregoing device, n, m and p are zero or an integer designating that such zones may or may not be present. The device comprises however, a plurality, i.e. at least two layers of different density. In addition, there may be more zones than those indicated, depending on the specific immunoassay being performed and the desired location of reagents.

The materials of Zone 1 et seq. have increasing specific gravities proceeding from the uppermost to the lowermost layers, so designed so that the lower specific gravities of unbound reactants in the assay system remain in the lower density layers while the reacted reagents settle into the higher density layers. The separation of these zones of different density can be enhanced by selecting materials that are poorly miscible with each other (eg. oil or oil-like materials and water wherein the oil may be of higher or lower density than the water) The concept of the invention is aided by the use of the magnet which attracts the reacted complex yielding a much faster settling of the reacted tagged reagents to the magnet area than would occur by simple gravity and elapsed time.

A general procedure for the invention utilizing the device generically described above is as follows:

1. Add magnetic particle tagged anti-human globulin (Mag-AHG) to the device, preferably at Zone 2.

2. Add sample or saline control to Zone 1. Sample depends on the specific test. being performed, i.e. direct Coombs, antibody screening, crossmatch, red cell typing or the like.

3. Add at Zone 1 the required reagents for the specific test being performed.

4. Allow the reagents to react and separate so that the cells enter Zone 2.

5. Optionally add a driving solution such as a buffer.

6. Allow the magnet to attract the complex formed by the Mag-AHG with any reacting antibody reagent, optionally moving the complex from a lower density layer to a higher density layer.

7. Read at magnet.

Another embodiment, especially when using liquid layers as zones in the above device, is:

A) Incubate the reactants of the test with the sample, such as RBCs, antibody, and a magnetically tagged universal RBC reactant, such as a lectin, i.e., Mag-lectin, in Zone 1.

B) Allow the reaction, if any, to take place.

C) Apply the magnetic field to allow the antibody/RBC/lectin Mag complex to pass through Zone 2, or Zone 3 if present, and be washed therein and to pass through a Zone of labeled AHG, for example enzyme labeled AHG, in a higher density Zone 3 or Zone 4. There the enzyme labeled AHG captures any reacted, sensitized RBC carrying reagent antibody. Unreacted AHG stays in its own density zone.

D) Under the influence of the magnet, the Mag complex particles descend further to the magnet leaving unreacted debris in the uppermost layers of lower density and leaving only the heavier reacted Mag tagged RBC's at the magnet demonstrating a positive. If the selected label requires a substrate conversion to a product to be visualized as does the enzyme label, the substrate is in this denser region.

E) Read the label of the reacted AHG or product produced at the magnet site.

MAGNET COATED WITH ANTIBODY EXAMPLE: Another embodiment is applicable to the direct testing for specific antigens on patient cells. Magnetic particles are coated with a specific antibody (for example Anti-A) and mixed with red cells which may or may not contain the A antigen. The magnetic particles react with the A antigen on the red cell and will pull those red cells to a magnet through more dense fluids, or alternatively if the fluids are flowing in a porous strip the red cells coated with magnetic particles will accumulate near the magnetic force field and those cells not coated with magnetic particles will be washed away as they flow by the magnet.

MAGNET COATED WITH LECTIN (ANTI-RBC) EXAMPLE: For detection of specific antibodies in patient's serum, for example detection of A,B antigens or O cells and Anti-A, Anti-B, Anti-AB respectively, the test is run in the same way antibody screening is performed. In either case, the reactants in the form of the magnetically tagged reagent cells or reagent antibodies are added directly onto Zone 1 and allowed to react therein and then pass into other Zones of higher density. Labeled anti-human globulin present in a lower zone is used to label any reacted complex for detection.

Briefly, the invention also applies to systems using antibody coated magnetic particles and magnets, to be applied to multiplexing cross-matching of many donors with patient serum in one reaction vessel, to antibody screening with multiple cells, to detecting fetal red blood cells in a sample of maternal blood and other applications where it is desirable to detect the presence of a minor population of red blood cells from another individual in a blood sample. In addition, the novel magnetic particle method can be applied to straight blood typing of single individual patients and donors, and to other blood cell serological (immunohematology) testing. According to a preferred embodiment, a vessel or an array of vessels such as a microtiter plate, or other segregated localized area or volume in other media, is provided that is designed to allow the red blood cells of the test sample to be contacted with a test solution having activated magnetic particles, which will attach to the test red blood cells that react with the test solution but not to test red blood cells that do not react with the solution.

A magnetic field, generated by a magnet or electromagnet, is selectively applied to the medium, which causes the red blood cells to be held in a manner allowing the red blood cells not decorated with magnetic particles to be removed or allowing the red blood cells held by the magnetic field to be removed. The red blood cells held by the magnetic field are re-suspended and quantitated spectrophotometrically. Enzyme amplification of the signal may be employed to increase sensitivity. In one preferred embodiment, the magnetic field is applied at the underside of a microplate in which the test solution is added to the mother's red blood cell suspension. The degree of magnetic force applied to the membrane may be selectively adjusted to vary the width or surface area of the capture line or zone.

METHODS FOR DETECTING AND QUANTIFYING FMH IN RH NEGATIVE MOTHERS: Because of the great sensitivity of the magnetic method, the general method can be specifically adapted to detect and quantitate fetal red cells in maternal blood, including blood samples from Rh-negative mothers. During pregnancy the blood circulations of mother and baby are separate and do not mix. However, harmless leakage of small amounts of blood from the baby's circulation into the mother's circulation is usual in almost every pregnancy. This is called Fetal Maternal Hemorrhage (FMH). Diagnostic tests to detect and measure the amount of baby's blood in the mother's blood sample are very important in the case of an Rh Negative mother pregnant with an Rh Positive baby. In these cases RBC leakage from Rh Positive fetus to Rh Negative mother, fetal maternal hemorrhage (FMH) occurs late in pregnancy and during delivery may cause Rh immunization of the mother and consequent Hemolytic Disease of the Fetus and Newborn in her future Rh-positive babies. It is very important to screen for and detect such occurrences.

The standard of care to prevent Rh iso-immunization and therefore Rh Hemolytic Disease is to administer RhoGAM® Ortho Pharmaceutical Co., Raritan, N.J., to all Rh Negative mothers at time of risk of Rh immunization, which is late pregnancy and delivery when FMH regularly occurs. One dose of RhoGAM, which covers FMH up to 15 ml, is given at 28 weeks gestation to all Rh negative mothers and another dose after delivery if the baby is Rh-positive. However, occasionally there may be a large and even massive FMH, which must be detected and measured because in that case multiple doses of RhoGAM are necessary to prevent Rh immunization.

It is standard to screen all Rh Negative mothers after delivery of an Rh Positive baby for FMH with a diagnostic screening test. If the screening test is positive it is necessary to quantitate the FMH so as to determine how many does of RhoGAM to give.

The current screening test is a commercially available kit which employs mixed field detection in which any baby's red blood cells in the mother's blood sample form “rosettes” which are seen under the microscope and counted by a technologist.

The currently used quantitative test for fetal RBC in mother's blood, the Kleihauer-Betke fetal cells stain and manual count, is used when the screening test is positive. It is sensitive to less than 0.1 ml of fetal cells in the mother's circulation and is quantitative. However the Kleihauer-Betke test is not a satisfactory test because it is manual, time consuming, requires skill and care, involves a technician training and competency assessment burden, uses unstable unpredictable reagents, is prone to false positive and false negative results and is very imprecise.

There is an unmet need for a FMH test that is rapid, economic and performs both screening and quantitative functions, and is objective in that it gives a numeric result free from the subjectivity of a technologist counting rosettes are in the average microscopic field.

The novel FMH method of the invention is simple, provides objective quantitative data and is easily adapted for automation. In this particular embodiment of the invention, specific antibody reagent tagged with magnetic particles, is applied in a liquid medium to a red blood cell sample in a vessel that contains presumably a minor population(s) of red cells from a different individual. The magnetically tagged antibodies bind to red blood cells that carry the chosen specific antigen on their surface, thus attaching magnetic particles to the red cell surface of antigen positive cells (of the minor population) but not to antigen negative cells (of the patient). Reagent magnetic antibody is carefully chosen to react with the minor population of red cells that is to be detected and measured so only the minor population of cells would be decorated with magnetic particles. A magnetic field is then applied and draws the tagged cells to the inside surface of the vessel directly, or through a separate zone of higher density to separate the unbound cells from the magnet tagged cells, or other solid state and immobilizes them there. This is possible where different blood groups can be identified on the transfused red blood cells but not on the patient's red blood cells.

In one embodiment of the FMH procedure, the magnetic particle test kit procedure would be as follows: incubate maternal blood with anti-D coated magnetic particles, apply magnetic field, remove free red blood cells physically or by separating them in a separate zone, pipette color developer reagent which would hemolyse residual fetal RBC and develop color. Read OD in capture zone using calorimeter. This procedure is done with a sample of blood collected from an Rh negative mother that has delivered an Rh positive baby. The key reagent is a suspension of magnetic particles (iron) that are coated with anti-D antibodies. These may be prepared by techniques well-known in the art. The concentration of the magnetic particles and mother's blood used in the assay will be important and need to be standardized, but in general there should be an excess of magnetic particles to the expected maximum fetal cells in the assay.

A preferred embodiment is to incubate mother's blood sample with magnetic anti-Rh reagent and start flow towards the magnet. If a “minor population” of Rh-positive fetal red cells were present in the mother's blood sample they will be captured by the magnet and the mother's Rh negative red cells will flow on past the magnet. The fetal cells are quantitated by measuring the amount or proportion of red cells stopped on magnet.

A specific procedure for an FMH determination is as follows:

To two test tubes (labeled 1 and 2) add an equal volume of a red cell suspension collected from the mother (note the volume and concentration of cells will be important). To two other tubes (labeled 3, and 4) add the same volume of the FMH calibrator. The volume added to each tube will be about 100 microliters. To one of the maternal (tube 2) and one of the calibrator tubes (tube 4) add the Mag-anti D reagent. The volume will be about 100 microliters of the reagent. Incubate all tubes approximately 5-30 minutes) with occasional mixing. Add 1 ml saline to tubes 2 and 4 and place them in the magnet apparatus. Wait 1 to 5 minutes to allow the magnetic particles to separate then decant unbound cells in tubes 2 and 4. Fill tubes 2 and 4 with saline (keeping them in the magnet apparatus, decant unbound cells. To tubes 1, 2, 3, and 4 add a lytic agent (1 ml water), compare the OD at appropriate wave length (540 nm). Compare the OD tube 2/OD tube 1 (TEST OD RATIO) with the OD tube 4/OD tube 3 (CONTROL OD RATIO), Divide the TEST OD RATIO by the CONTROL OD RATIO to determine the number of RhoGam Doses needed. A similar procedure can be designed where the red cells and magnets are placed in a test tube containing a solution more dense than the red cell suspension so that red cells can not readily settle through this more dense zone. After the red cells react with the magnetic particles, a magnetic force field pulls the magnetic particles and any red cells attached to the through the denser zone and to the portion of the device where these magnetic bound cells can be quantitated. It is simple to compare the number of separated magnetic tagged cells to the total number of cells or the untagged cells.

Besides the test for FMH, there are other blood bank laboratory and forensic laboratory applications where it is important to detect the presence of a minor population of red blood cells from a second individual in a sample of blood belonging to the first individual, such as athlete blood doping. This test is valuable in assaying the survival of transfused blood from various donors, in the biological compatibility test, multiplexing cross-matching of many donors with a patient(s) in one reaction vessel, antibody screening with multiple cells, research investigation of rare red blood cell chimeras and other special situations, in addition to detecting fetal red blood cells in a sample of maternal blood from an Rh Negative mother.

As a statement of general applicability, in selecting the various densities for the assay construct of the present invention, the specific gravity of the uppermost layer of serum addition should be equal to or greater than the specific gravity of serum and the specific gravity of the soluble materials in the sample reaction mixture. Once the cells, serum, and Mag particles are mixed, the specific gravity of the soluble materials may decrease slightly helping to keep the layer with the sample in place at the top. Reaction products will drop to the layers of like densities. Subsequent lower zones have a specific gravity higher than the upper zone.

This general method can be applied to all of the clinical situations listed above where a need for detection and quantitation of a mixed field of red blood cells in a sample is required. Finally the method is applicable to performing blood typing and detection of anti-red cell antibodies on the blood of a single individual where no mixture exists.

What is claimed:

1. The method for detecting the presence or absence of an antigen on a blood cell carrier which comprises:
1. providing in an assay construct, a mixture of:
a) a blood cell sample suspected of carrying said antigen to be determined,
b) an antibody specific for the antigen sought to be determined, and
c) a magnetic particle tagged moiety which is reactable with said blood cell, to form a first complex of a), b) and c) above, if said antigen is present,
2. reacting said first complex with a labeled anti-human globulin (AHG) to form a second complex of said first complex and said labeled AHG wherein said AHG is attached to the antibody portion of said first complex if said antibody portion is present, and wherein said label is non-magnetic,
3. applying a magnetic field from a magnet to said second complex whereby said second complex is attracted to said magnet and thereby is separated from unreacted labeled AHG, and
4. reading the label, or a conversion product of said label, of the complexed AHG in the second complex to determine the presence or absence of said antigen sought to be determined.

2. The method for detecting the presence or absence of an antibody which comprises:
1. providing in an assay construct, a mixture of:
a) a sample suspected of containing an antibody to be determined,
b) a red blood cell carrying an antigen specific for the antibody sought to be determined,
c) a magnetic particle tagged moiety which is reactable with said red blood cell,
to form a first complex of a), b) and c) above, if said antibody is present,
2. reacting said first complex with a labeled anti-human globulin (AHG) to form form a second complex of said first complex and said labeled AHG wherein said AHG is attached to the antibody portion of said first complex if said antibody portion is present, and wherein said label is non-magnetic,
3. applying a magnetic field from a magnet to said second complex whereby said second complex is attracted to said magnet and thereby is separated from unreacted labeled AHG, and
4. reading the label, or a conversion product of said label, of the complexed AHG in the second complex to determine the presence or absence of said antigen sought to be determined.

3. The method for detecting the presence or absence of an antigen on a blood cell carrier which comprises:
1. providing in an assay construct, a mixture of:
a) a blood cell sample suspected of carrying said antigen to be determined, and
b) an antibody specific for the antigen sought to be determined, said antibody being tagged with magnetic particles, and
forming a complex of a) and b) above, if said antigen is present, wherein said assay construct comprises layers having different densities from one another, and wherein said mixture is provided at the lower density layer,
2. applying a magnetic field from a magnet to said mixture whereby said complex, if any, and any unreacted magnetic specific antibody, if any, is attracted to said magnet and thereby is separated from non-magnetic entities,
3. reading the presence or absence of hemoglobin in the attracted mixture to determine the presence or absence of said antigen sought to be determined.

4. The method of claim 1 wherein the blood cell carrier is a red blood cell.

5. The method of claim 1 wherein the magnetic tagged moiety is a lectin.

6. The method of claim 1 wherein the label of the antihuman globulin is an enzyme.

7. The method of claim 1 wherein the assay construct comprises at least two layers having different densities.

8. The method of claim 7 wherein there is at least one aqueous layer and at least one water immiscible layer in said construct.

9. The method of claim 8 wherein the water immiscible layer is of lower density than water.

10. The method of claim 8 wherein the water immiscible layer is of higher density than water.

11. The method of claim 1 wherein the specific antibody is anti-A, anti-B, or anti-D.

12. The method according to claim 7 wherein between steps 1 and 2, the first complex is moved by the magnet from a lower density layer to a higher density layer comprising the labeled AHG.

13. The method according to claim 12 wherein step 3 moves the second complex to a higher density layer than the layer in which said second complex originated.

14. The method of claim 2 wherein the magnetic tagged moiety is a lectin.

15. The method of claim 2 wherein the label of the antihuman globulin is an enzyme.

16. The method of claim 2 wherein the assay construct comprises at least two layers having different densities.

17. The method of claim 16 wherein there is at least one aqueous layer and at least one water immiscible layer.

18. The method of claim 17 wherein the water immiscible layer is of lower density than water.

19. The method of claim 17 wherein the water immiscible layer is of higher density than water.

20. The method of claim 12 wherein the specific antibody is sought to be determined anti-A, anti-B, or anti-D.

21. The method of claim 3 wherein the blood cell carrier is a red blood cell.

22. The method of claim 3 wherein the magnetic tagged moiety is a lectin.

23. The method of claim 3 wherein there is at least one aqueous layer and at least one water immiscible layer.

24. The method of claim 3 wherein the assay construct comprises at least two layers having different densities.

25. The method of claim 23 wherein the water immiscible layer is of lower density than water.

24. The method of claim 23 wherein the water immiscible layer is of higher density than water.

25. The method of claim 3 wherein the specific antibody is Anti-A, Anti-B, or Anti-D.

26. The method of claim 3 performed for determining fetal maternal hemorrhage wherein the antibody tagged with the magnetic particles is Anti-D.

27. The method of claim 2 for performing a crossmatch of a donor's red cells with patient's serum.
